(19) United States
(12) Patent Application Publication
(10) Pub. No.: US 2022/0098184 A1
(43) Pub. Date: Mar. 31, 2022

(54) SOLID DISPERSION FORMULATIONS OF AN FXR AGONIST

(71) Applicant: Terms Pharmaceuticals, Inc., Foster City, CA (US)

(72) Inventors: Yujin WANG, Foster City, CA (US); Chutian Shu, Foster City, CA (US); Sheng Guo, Foster City, CA (US); Matt Duan, Foster City, CA (US); Jianwei Bian, Foster City, CA (US); Jianxin Deng, Foster City, CA (US); Aiyong Si, Foster City, CA (US)

(21) Appl. No.: 17/447,397

(22) Filed: Sep. 10, 2021

(30) Foreign Application Priority Data
Sep. 11, 2020 (CN) PCT/CN2020/114782

(51) Int. Cl. C07D 413/14; A61K 31/454; A61K 47/32

(52) U.S. Cl. CPC C07D 413/14; A61K 47/32; A61K 31/454

(57) ABSTRACT

Provided herein are pharmaceutical compositions comprising a substantially amorphous form of 6-(4-((5-cyclopropyl-3-(2,6-dichlorophenyl)isoxazol-4-yl)methoxy)piperidin-1-yl)-1-methyl-1H-indole-3-carboxylic acid, such as solid dispersions of 6-(4-((5-cyclopropyl-3-(2,6-dichlorophenyl)isoxazol-4-yl)methoxy)piperidin-1-yl)-1-methyl-1H-indole-3-carboxylic acid; methods of preparation thereof, and methods of use thereof.

### CROSS-REFERENCE TO RELATED APPLICATIONS

This application claims priority to and the benefit of International Application No.: PCT/CN2020/114782, filed on Sep. 11, 2020, the disclosure of which is hereby incorporated herein by reference in its entirety for all purposes.

### FIELD

Provided herein are solid dispersions of 6-(4-((5-cyclopropyl-3-(2,6-dichlorophenyl)isoxazol-4-yl)methoxy)piperidin-1-yl)-1-methyl-1H-indole-3-carboxylic acid, pharmaceutic compositions thereof, methods of preparation thereof, and methods of use thereof.

### BACKGROUND

Therapeutic agents that function as farnesoid X receptor (FXR) agonists have the potential to remedy or improve the lives of patients in need of treatment of liver disorders such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). U.S. Pat. No. 8,153,624, the content of which is incorporated herein by reference in its entirety, discloses 6-(4-((5-cyclopropyl-3-(2,6-dichlorophenyl)isoxazol-4-yl)methoxy)piperidin-1-yl)-1-methyl-1H-indole-3-carboxylic acid (designated herein as Compound I), which has the structure shown below.

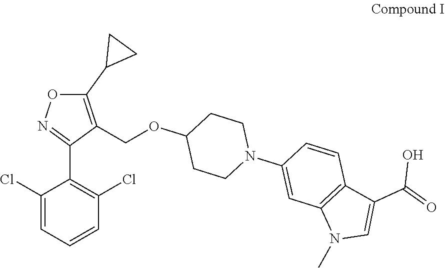 [SMILES: Cn1cc(C(=O)O)c2ccc(N3CCC(OCc4c(-c5c(Cl)cccc5Cl)noc4C4CC4)CC3)cc21]

Compound I is a potent FXR agonist being developed as a therapeutic for liver disorders. However, Compound I is a BCS class II compound with low aqueous solubility. Therefore, there remains a need for Compound I formulations with improved pharmacokinetic properties.

### BRIEF SUMMARY

In one aspect, provided herein are solid dispersions comprising Compound I.

In another aspect, provided herein are methods of preparing solid dispersions comprising Compound I.

In another aspect, provided herein are methods of treating a subject in need of treatment of liver disorders using solid dispersions comprising Compound I.

### DESCRIPTION OF THE DRAWINGS

FIG. 1 shows AUC₀₋ₗₐₛₜ of Compound I following oral administration of Compound I Capsules and Solid dispersion Tablets in Dogs (Meant±SD, n=8). Special fed: Dogs were fed with standard canine food 30 minutes prior to test article dosing; fasted: dogs were fasted overnight, and food returned about 4 hour post-dose; pentagastrin treatment: intramuscular injection of pentagastrin at 6 μg/kg 15 minuets prior to test article dosing; famotidine treatment: famotidine was administered at 20 mg/dog 30 minutes prior to test article dosing.

### DETAILED DESCRIPTION

### Definitions

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in connection with a value, contemplate a variation within ±15%, within ±10%, within ±5%, within ±4%, within ±3%, within ±2%, within ±1%, or within ±0.5% of the specified value. Reference to “about” a value or parameter herein includes (and describes) embodiments that are directed to that value or parameter per se. For example, description referring to “about X” includes description of “X”.

As used herein, the term “solid dispersion” refers to a composition in a solid state comprising at least two components, wherein one component is dispersed in the other component or components. For example, a therapeutic agent may be dispersed in a matrix comprising a polymer.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, the term “excipient” refers to an inactive substance with which the active ingredient is administered. Examples of excipients include, but are not limited to, glidants, diluents, disintegrants, lubricants, or vehicles.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

As used herein, the term “subject” refers to an animal, including, but are not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, the term “therapeutically effective amount” refers to an amount of a compound or composition sufficient to treat a specified disorder, condition or disease such as to ameliorate, to palliate, to lessen, and/or to delay one or more of its symptoms.

As used herein, the term “substantially free of” means that the composition contains the indicated substance or substances in an amount of less than about 25%, less than about 20%, less than about 15%, less than about 10%, less than about 5%, less than about 4%, less than about 3%, less than about 2%, or less than about 1% by weight.

### Solid Dispersions

In one aspect, provided is a solid dispersion comprising Compound I having the formula:

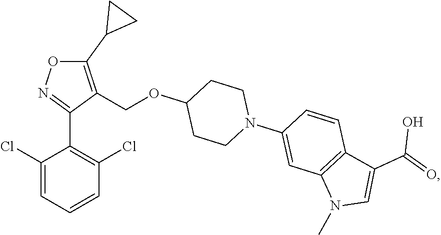 [SMILES: Cn1cc(C(=O)O)c2ccc(N3CCC(OCc4c(-c5c(Cl)cccc5Cl)noc4C4CC4)CC3)cc21]

wherein the compound is substantially amorphous and is dispersed in a polymer.

As used herein, the term “substantially amorphous” means that more than about 50%, more than about 60%, more than about 70%, more than about 75%, more than about 80%, more than about 85%, more than about 90%, more than about 95%, more than about 96%, more than about 97%, more than about 98%, more than about 99%, or more than about 99.9% by weight of Compound I in the solid dispersion is amorphous. In some embodiments, more than about 90% of Compound I in the solid dispersion is amorphous. In some embodiments, more than about 95% by weight of Compound I in the solid dispersion is amorphous. In some embodiments, more than about 96% by weight of Compound I in the solid dispersion is amorphous. In some embodiments, more than about 97% by weight of Compound I in the solid dispersion is amorphous. In some embodiments, more than about 98% by weight of Compound I in the solid dispersion is amorphous. In some embodiments, more than about 99% by weight of Compound I in the solid dispersion is amorphous. In some embodiments, more than about 99.9% by weight of Compound I in the solid dispersion is amorphous. In some embodiments, the solid dispersion is substantially free of a crystalline form of Compound I.

In some embodiments, the polymer is a hydrophilic polymer. Examples of hydrophilic polymers include, but are not limited to, homopolymers and copolymers of vinyl lactams (e.g., homopolymers and copolymers of vinylpyrrolidone or vinylcaprolactam); polyethylene glycols; celluloses, cellulose esters and cellulose ethers (e.g., methylcellulose, hydroxypropyl methylcellulose, hydroxypropyl methylcellulose acetate succinate, and hydroxypropyl methylcellulose phthalate); polyacrylates (e.g., ammonio methacrylate copolymer and polyacrylic acid); and mixtures thereof.

In some embodiments, the polymer is a homopolymer or a copolymer of a vinyl lactam. In some embodiments, the polymer is a homopolymer or a copolymer of vinylpyrrolidone or vinylcaprolactam. In some embodiments, the polymer is a homopolymer or a copolymer of vinylpyrrolidone (e.g., poly(vinylpyrrolidone) or vinyl pyrrolidone-vinyl acetate copolymer). In some embodiments, the polymer is a vinylpyrrolidone-vinyl acetate copolymer (e.g., the product sold under the trademark Kollidon® VA64). In some embodiments, the polymer is a homopolymer or a copolymer of vinylcaprolactam (e.g., poly(vinylcaprolactam) or vinylcaprolactam-vinyl acetate-ethylene glycol copolymer). In some embodiments, the polymer is vinylcaprolactam-vinyl acetate-ethylene glycol copolymer (e.g., the product sold under the trademark Soluplus®). In some embodiments, the polymer is vinylpyrrolidone-vinyl acetate copolymer or vinylcaprolactam-vinyl acetate-ethylene glycol copolymer. In some embodiments, the vinylcaprolactam-vinyl acetate-ethylene glycol copolymer is a vinylcaprolactam-vinyl acetate-ethylene glycol graft copolymer.

In some embodiments, the weight ratio of Compound I to the polymer is between about 1:1 and about 1:20, between about 1:1 and about 1:15, between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, or between about 1:1 and about 1:2. In some embodiments, the weight ratio of Compound I to the polymer is about 1:1, about 1:2, about 1:3, about 1:4, about 1:5, about 1:6, about 1:7, about 1:8, about 1:9, about 1:10, about 1:15, or about 1:20. In some embodiments, the weight ratio of Compound I to the polymer is between about 1:1 and about 1:10. In some embodiments, the weight ratio of Compound I to the polymer is about 1:3.

### Pharmaceutical Compositions

In another aspect, provided is a pharmaceutical composition comprising a solid dispersion disclosed herein and a pharmaceutically acceptable excipient.

In some embodiments, the pharmaceutical composition disclosed herein is administered by oral administration. In some embodiments, the pharmaceutical composition is formulated for immediate release. In some embodiments, the pharmaceutical composition is formulated for sustained release. In some embodiments, the pharmaceutical composition is in the form of a tablet or a capsule. In some embodiments, the pharmaceutical composition is in the form of a tablet. In some embodiments, the tablet is coated. In some embodiments, the pharmaceutical composition is in the form of a capsule.

In some embodiments, the pharmaceutically acceptable excipient comprises a diluent. As used herein, the term “diluent” refers to a substance that is used to dilute an active ingredient prior to delivery. Diluents can also serve to stabilize the active ingredient. Examples of diluents include, but are not limited to, starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose monohydrate, dicalcium phosphate, compressible sugars, dibasic calcium phosphate dehydrate, mannitol, and tribasic calcium phosphate. In some embodiments, the diluent comprises microcrystalline cellulose. In some embodiments, the pharmaceutical composition comprises the diluent in the amount of between about 10% and about 90%, between about 10% and about 80%, between about 10% and about 70%, between about 10% and about 60%, between about 10% and about 50%, between about 10% and about 40%, between about 10% and about 30%, between about 10% and about 20%, between about 20% and about 90%, between about 20% and about 80%, between about 20% and about 70%, between about 20% and about 60%, between about 20% and about 50%, between about 20% and about 40%, between about 20% and about 30%, between about 30% and about 90%, between about 30% and about 80%, between about 30% and about 70%, between about 30% and about 60%, between about 30% and about 50%, between about 30% and about 40%, between about 40% and about 90%, between about 40% and about 80%, between about 40% and about 70%, between about 40% and about 60%, between about 40% and about 50%, between about 50% and about 90%, between about 50% and about 80%, between about 50% and about 70%, between about 50% and about 60%, between about 60% and about 90%, between about 60% and about 80%, between about 60% and about 70%, between about 70% and about 90%, between about 70% and about 80%, or about 80% and about 90% by weight. In some embodiments, the pharmaceutical composition comprises the diluent in the amount of about 10%, about 15%, about 20%, about 25%, about 30%, about 35%, about 40%, about 45%, about 50%, about 55%, about 60%, about 65%, about 70%, about 75%, about 80%, about 85%, or about 90% by weight. In some embodiments, the pharmaceutical composition comprises the diluent in the amount of about 10% by weight. In some embodiments, the pharmaceutical composition comprises the diluent in the amount of about 15% by weight. In some embodiments, the pharmaceutical composition comprises the diluent in the amount of about 20% by weight. In some embodiments, the pharmaceutical composition comprises the diluent in the amount of about 25% by weight. In some embodiments, the pharmaceutical composition comprises the diluent in the amount of about 30% by weight. In some embodiments, the pharmaceutical composition comprises the diluent in the amount of about 35% by weight. In some embodiments, the pharmaceutical composition comprises the diluent in the amount of about 40% by weight. In some embodiments, the pharmaceutical composition comprises the diluent in the amount of about 45% by weight. In some embodiments, the pharmaceutical composition comprises the diluent in the amount of about 50% by weight. In some embodiments, the pharmaceutical composition comprises the diluent in the amount of about 55% by weight. In some embodiments, the pharmaceutical composition comprises the diluent in the amount of about 60% by weight. In some embodiments, the pharmaceutical composition comprises the diluent in the amount of about 65% by weight. In some embodiments, the pharmaceutical composition comprises the diluent in the amount of about 70% by weight. In some embodiments, the pharmaceutical composition comprises the diluent in the amount of about 75% by weight. In some embodiments, the pharmaceutical composition comprises the diluent in the amount of about 80% by weight. In some embodiments, the pharmaceutical composition comprises the diluent in the amount of about 85% by weight. In some embodiments, the pharmaceutical composition comprises the diluent in the amount of about 90% by weight.

In some embodiments, the pharmaceutically acceptable excipient comprises a disintegrant. As used herein, the term “disintegrant” refers to a substance which, upon addition to a solid formulation, facilitates its break-up or disintegration after administration and permits the release of an active ingredient as efficiently as possible to allow for its rapid dissolution. Examples of disintegrants include, but are not limited to, maize starch, sodium starch glycolate, croscarmellose sodium, crospovidone, microcrystalline cellulose, modified corn starch, sodium carboxymethyl starch, povidone, pregelatinized starch, and alginic acid. In some embodiments, the disintegrant comprises crospovidone. In some embodiments, the pharmaceutical composition comprises the disintegrant in the amount of between about 10% and about 50%, between about 10% and about 40%, between about 10% and about 30%, between about 10% and about 20%, between about 20% and about 50%, between about 20% and about 40%, between about 20% and about 30%, between about 30% and about 50%, between about 30% and about 40%, or between about 40% and about 50% by weight. In some embodiments, the pharmaceutical composition comprises the disintegrant in the amount of about 10%, about 15%, about 20%, about 25%, about 30%, about 35%, about 40%, about 45%, or about 50% by weight. In some embodiments, the pharmaceutical composition comprises the disintegrant in the amount of about 10% by weight. In some embodiments, the pharmaceutical composition comprises the disintegrant in the amount of about 15% by weight. In some embodiments, the pharmaceutical composition comprises the disintegrant in the amount of about 20% by weight. In some embodiments, the pharmaceutical composition comprises the disintegrant in the amount of about 25% by weight. In some embodiments, the pharmaceutical composition comprises the disintegrant in the amount of about 30% by weight. In some embodiments, the pharmaceutical composition comprises the disintegrant in the amount of about 35% by weight. In some embodiments, the pharmaceutical composition comprises the disintegrant in the amount of about 40% by weight. In some embodiments, the pharmaceutical composition comprises the disintegrant in the amount of about 45% by weight. In some embodiments, the pharmaceutical composition comprises the disintegrant in the amount of about 50% by weight.

In some embodiments, the pharmaceutically acceptable excipient comprises a glidant. As used herein, the term “glidant” refers to a substance used in tablet and capsule formulations to improve flow-properties during tablet compression and to produce an anti-caking effect. Examples of glidants include, but are not limited to, colloidal silicon dioxide, talc, fumed silica, starch, starch derivatives, and bentonite. In some embodiments, the glidant comprises colloidal silicon dioxide. In some embodiments, the pharmaceutical composition comprises the glident in the amount of about 0.1% and about 1% by weight. In some embodiments, the pharmaceutical composition comprises the glident in the amount of about 0.1%, about 0.2%, about 0.3%, about 0.4%, about 0.5%, about 0.6%, about 0.7%, about 0.8%, about 0.9%, or about 1% by weight. In some embodiments, the pharmaceutical composition comprises the glident in the amount of about 0.1% by weight. In some embodiments, the pharmaceutical composition comprises the glident in the amount of about 0.2% by weight. In some embodiments, the pharmaceutical composition comprises the glident in the amount of about 0.3% by weight. In some embodiments, the pharmaceutical composition comprises the glident in the amount of about 0.4% by weight. In some embodiments, the pharmaceutical composition comprises the glident in the amount of about 0.5% by weight. In some embodiments, the pharmaceutical composition comprises the glident in the amount of about 0.6% by weight. In some embodiments, the pharmaceutical composition comprises the glident in the amount of about 0.7% by weight. In some embodiments, the pharmaceutical composition comprises the glident in the amount of about 0.8% by weight. In some embodiments, the pharmaceutical composition comprises the glident in the amount of about 0.9% by weight. In some embodiments, the pharmaceutical composition comprises the glident in the amount of about 1% by weight.

In some embodiments, the pharmaceutically acceptable excipient comprises a lubricant. As used herein, the term “lubricant” refers to a substance which is added to a powder blend to prevent the compacted powder mass from sticking to the equipment during the tableting or encapsulation process. Examples of lubricants include, but are not limited to, magnesium stearate, stearic acid, silica, fats, talc, solubilizers such as fatty acids (e.g., lauric acid and oleic acid). In some embodiments, the lubricant comprises magnesium stearate. In some embodiments, the pharmaceutical composition comprises the lubricant in the amount of about 0.1% and about 1% by weight. In some embodiments, the pharmaceutical composition comprises the lubricant in the amount of about 0.1%, about 0.2%, about 0.3%, about 0.4%, about 0.5%, about 0.6%, about 0.7%, about 0.8%, about 0.9%, or about 1% by weight. In some embodiments, the pharmaceutical composition comprises the lubricant in the amount of about 0.1% by weight. In some embodiments, the pharmaceutical composition comprises the lubricant in the amount of about 0.2% by weight. In some embodiments, the pharmaceutical composition comprises the lubricant in the amount of about 0.3% by weight. In some embodiments, the pharmaceutical composition comprises the lubricant in the amount of about 0.4% by weight. In some embodiments, the pharmaceutical composition comprises the lubricant in the amount of about 0.5% by weight. In some embodiments, the pharmaceutical composition comprises the lubricant in the amount of about 0.6% by weight. In some embodiments, the pharmaceutical composition comprises the lubricant in the amount of about 0.7% by weight. In some embodiments, the pharmaceutical composition comprises the lubricant in the amount of about 0.8% by weight. In some embodiments, the pharmaceutical composition comprises the lubricant in the amount of about 0.9% by weight. In some embodiments, the pharmaceutical composition comprises the lubricant in the amount of about 1% by weight.

It is to be understood that a pharmaceutically acceptable excipient may contain a diluent, a disintegrant, a glidant, and/or a lubricant. As an example, in some embodiments, the pharmaceutical composition comprises a solid dispersion disclosed herein, a diluent such as microcrystalline cellulose, a disintegrant such as crospovidone, a glidant such as colloidal silicon dioxide, and a lubricant such as magnesium stearate.

In some embodiments, the pharmaceutical composition comprises Compound I in the amount of between about 1 mg and about 30 mg, between about 1 mg and about 25 mg, between about 1 mg and about 20 mg, between about 1 mg and about 15 mg, between about 1 mg and about 10 mg, between about 1 mg and about 5 mg, between about 5 mg and about 30 mg, between about 5 mg and about 25 mg, between about 5 mg and about 20 mg, between about 5 mg and about 15 mg, between about 5 mg and about 10 mg, between about 10 mg and about 30 mg, between about 10 mg and about 25 mg, between about 10 mg and about 20 mg, between about 10 mg and about 15 mg, between about 15 mg and about 30 mg, between about 15 mg and about 25 mg, between about 15 mg and about 20 mg, between about 20 mg and about 30 mg, or between about 25 mg and about 30 mg. In some embodiments, the pharmaceutical composition comprises about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, or about 30 mg of Compound I. In some embodiments, the pharmaceutical composition comprises about 5 mg or about 25 mg of Compound I. In some embodiments, the pharmaceutical composition comprises about 5 mg of Compound I. In some embodiments, the pharmaceutical composition comprises about 25 mg of Compound I.

In some embodiments, the pharmaceutical composition comprises a solid dispersion disclosed herein in the amount of between about 1% and about 50%, between about 1% and about 40%, between about 1% and about 30%, between about 1% and about 20%, between about 1% and about 10%, between about 1% and about 5%, between about 10% and about 50%, between about 10% and about 40%, between about 10% and about 30%, between about 10% and about 20%, between about 20% and about 50%, between about 20% and about 40%, between about 20% and about 30%, between about 30% and about 50%, between about 30% and about 40%, or between about 40% and about 50% by weight. In some embodiments, the pharmaceutical composition comprises a solid dispersion disclosed herein in the amount of about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% by weight. In some embodiments, the pharmaceutical composition comprises the solid dispersion in the amount of about 1% by weight. In some embodiments, the pharmaceutical composition comprises the solid dispersion in the amount of about 2% by weight. In some embodiments, the pharmaceutical composition comprises the solid dispersion in the amount of about 3% by weight. In some embodiments, the pharmaceutical composition comprises the solid dispersion in the amount of about 4% by weight. In some embodiments, the pharmaceutical composition comprises the solid dispersion in the amount of about 5% by weight. In some embodiments, the pharmaceutical composition comprises the solid dispersion in the amount of about 6% by weight. In some embodiments, the pharmaceutical composition comprises the solid dispersion in the amount of about 7% by weight. In some embodiments, the pharmaceutical composition comprises the solid dispersion in the amount of about 8% by weight. In some embodiments, the pharmaceutical composition comprises the solid dispersion in the amount of about 9% by weight. In some embodiments, the pharmaceutical composition comprises the solid dispersion in the amount of about 10% by weight. In some embodiments, the pharmaceutical composition comprises the solid dispersion in the amount of about 15% by weight. In some embodiments, the pharmaceutical composition comprises the solid dispersion in the amount of about 20% by weight. In some embodiments, the pharmaceutical composition comprises the solid dispersion in the amount of about 25% by weight. In some embodiments, the pharmaceutical composition comprises the solid dispersion in the amount of about 30% by weight. In some embodiments, the pharmaceutical composition comprises the solid dispersion in the amount of about 35% by weight. In some embodiments, the pharmaceutical composition comprises the solid dispersion in the amount of about 40% by weight. In some embodiments, the pharmaceutical composition comprises the solid dispersion in the amount of about 45% by weight. In some embodiments, the pharmaceutical composition comprises the solid dispersion in the amount of about 50% by weight.

It is to be understood that two or more values may combined. Thus, it is to be understood that any weight percentage value for the solid dispersion may be combined with any weight percentage value for the diluent, any weight percentage value for the disintegrant, any weight percentage value for the glidant, any weight percentage value for the lubricant as if each and every combination were specifically and individually listed.

### Methods of Preparing Solid Dispersions

In another aspect, provided herein is a method of preparing a solid dispersion disclosed herein. Techniques known in the art for preparing solid dispersions may be used. Examples of techniques that can be used include, but are not limited to, hot-melt extrusion and spray-drying.

In some embodiments, provided is a method of preparing a solid dispersion disclosed herein, comprising hot-melt extruding a mixture of Compound I and a polymer. In some embodiments, Compound I is in a crystalline form before extrusion. In some embodiments, Compound I is in a non-crystalline form before extrusion. In some embodiments, the polymer is a homopolymer or a copolymer of vinylpyrrolidone or vinylcaprolactam. In some embodiments, the polymer is a homopolymer or a copolymer of vinylpyrrolidone (e.g., poly(vinylpyrrolidone) or vinyl pyrrolidone-vinyl acetate copolymer). In some embodiments, the polymer is vinylpyrrolidone-vinyl acetate copolymer. In some embodiments, the polymer is a homopolymer or a copolymer of vinylcaprolactam (e.g., poly(vinylcaprolactam) or vinylcaprolactam-vinyl acetate-ethylene glycol copolymer). In some embodiments, the polymer is vinylcaprolactam-vinyl acetate-ethylene glycol copolymer. In some embodiments, the vinylcaprolactam-vinyl acetate-ethylene glycol copolymer is a vinylcaprolactam-vinyl acetate-ethylene glycol graft copolymer. In some embodiments, the hot-melt extrusion is performed at a temperature of between about 120° C. and about 180° C. In some embodiments, the hot-melt extrusion is performed at a temperature of about 120° C., about 125° C., about 130° C., about 135° C., about 140° C., about 145° C., about 150° C., about 155° C., about 160° C., about 165° C., about 170° C., about 175° C., or about 180° C.

### Methods of Use

In another aspect, provided herein is a method of treating a liver disorder in a patient (e.g., a human patient) in need thereof comprising administering a therapeutically effective amount of a solid dispersion disclosed herein. In some embodiments, the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is NAFLD or NASH. In some embodiments, the liver disorder is NAFLD. In some embodiments, the liver disorder is NASH. In some embodiments, the patient has had a liver biopsy. In some embodiments, the method further comprises obtaining the results of a liver biopsy.

In some embodiments, provided is a method of impeding or slowing the progression of NAFLD to NASH in a patient (e.g., a human patient) in need thereof comprising administering a therapeutically effective amount of a solid dispersion disclosed herein.

Compound I is preferentially distributed to the liver, which, without being bound by theory, would allow the compound to reach its FXR target in the liver with fewer off-target adverse effects. For example, Compound I has an approximately 20-fold higher concentration in the liver than in the plasma, kidney, lungs, heart, and skin. This trait would likely be particularly beneficial for vulnerable populations, such as children, the elderly, and people with comorbidities.

Further, pruritus is a well-documented adverse effect of several FXR agonists and can result in patient discomfort, a decrease in patient quality of life, and an increased likelihood of ceasing treatment. Pruritus is particularly burdensome for indications, such as those described herein, including NASH, for which chronic drug administration is likely. The tissue specificity of Compound I, in particular the preference for liver over skin tissue is a striking and unpredicted observation that makes it more likely that the compound will not cause pruritus in the skin, a theory that has been substantiated by human trials thus far.

In some embodiments, provided is a method of treating a liver disorder in a patient in need thereof (e.g., a human patient) with Compound I that preferentially distributes in liver tissue over one or more of kidney, lung, heart, and skin tissues, the method comprising administering a therapeutically effective amount of Compound I, wherein Compound I is formulated as a solid dispersion disclosed herein.

In some embodiments, provided herein is a method of treating a liver disorder in a patient in need thereof comprising administering a therapeutically effective amount of Compound I, wherein Compound I is formulated as a solid dispersion disclosed herein and wherein Compound I does not activate TGR5 signaling. In some embodiments, the level of an FXR-regulated gene is increased. In some embodiments, the level of small heterodimer partner (SHP), bile salt export pump (BSEP) and fibroblast growth factor 19 (FGF-19) is increased. In some embodiments, the liver disorder is NASH.

In some embodiments, provided herein is a method of reducing liver damage comprising administering a therapeutically effective amount of Compound I, wherein Compound I is formulated as a solid dispersion disclosed herein, to an individual in need thereof. In some embodiments, fibrosis is reduced. In some embodiments, the level of expression of one or more markers for fibrosis is reduced. In some embodiments, the level of Ccr2, Col1a1, Col1a2, Col1a3, Cxcr3, Dcn, Hgf, Il1a, Inhbe, Lox, Loxl1, Loxl2, Loxl3, Mmp2, pdgfb, Plau, Serpine1, Perpinh1, Snai, Tgfb1, Tgfb3, Thbs1, Thbs2, Timp2, and/or Timp3 expression is reduced. In some embodiments, the level of collagen is reduced. In some embodiments, the level of collagen fragments is reduced. In some embodiments, the level of expression of the fibrosis marker is reduced at least 2, at least 3, at least 4, or at least 5-fold. In some embodiments, the level of expression of the fibrosis marker is reduced about 2-fold, about 3-fold, about 4-fold, or about 5-fold.

In some embodiments, provided herein is a method of reducing liver damage comprising administering a therapeutically effective amount of Compound I, wherein Compound I is formulated as a solid dispersion disclosed herein, to an individual in need thereof. In some embodiments, inflammation is reduced. In some embodiments, one or more markers of inflammation are reduced. In some embodiments, the level of expression of Adgre1, Ccr2, Ccr5, Il1A, and/or Tlr4 is reduced. In some embodiments, the level of expression of the inflammation marker is reduced at least 2-, at least 3-, at least 4-, or at least 5-fold. In some embodiments, the level of expression of the fibrosis marker is reduced about 2-fold, about 3-fold, about 4-fold, or about 5-fold.

In some embodiments, the administration does not result in pruritus in the patient greater than Grade 2 in severity. In some embodiments, the administration does not result in pruritus in the patient greater than Grade 1 in severity. In some embodiments, the administration does not result in pruritus in the patient. The grading of adverse effects is known. According to Version 5 of the Common Terminology Criteria for Adverse Events (published Nov. 27, 2017), Grade 1 pruritus is characterized as “Mild or localized; topical intervention indicated.” Grade 2 pruritus is characterized as “Widespread and intermittent; skin changes from scratching (e.g., edema, papulation, excoriations, lichenification, oozing/crusts); oral intervention indicated; limiting instrumental ADL.” Grade 3 pruritus is characterized as “Widespread and constant; limiting self care ADL or sleep; systemic corticosteroid or immunosuppressive therapy indicated.” Activities of daily living (ADL) are divided into two categories: “Instrumental ADL refer to preparing meals, shopping for groceries or clothes, using the telephone, managing money, etc.,” and “Self care ADL refer to bathing, dressing and undressing, feeding self, using the toilet, taking medications, and not bedridden.”

Accordingly, in some embodiments, provided is a method of treating a liver disorder in a patient (e.g., a human patient) in need thereof with Compound I that does not result in detectable pruritus in the patient, the method comprising administering to the patient in need thereof a therapeutically effective amount of Compound I, wherein Compound I is formulated as a solid dispersion disclosed herein.

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, or 90 or more.

NAFLD and NASH are common causes of liver transplantation, but patients that already received one liver transplant often develop NAFLD and/or NASH again. Accordingly, in some embodiments, the patient has had a liver transplant.

In some embodiments, the patient's alkaline phosphatase, gamma-glutamyl transferase (GGT), alanine aminotransferase (ALT) and/or aspartate aminotransferase (AST) levels are elevated. In some embodiments, the GGT, ALT, and/or AST levels are elevated prior to treatment with a solid dispersion disclosed herein. In some embodiments, the patient's ALT level is about 2-4-fold greater than the upper limit of normal levels. In some embodiments, the patient's AST level is about 2-4-fold greater than the upper limit of normal levels. In some embodiments, the patient's GGT level is about 1.5-3-fold greater than the upper limit of normal levels. In some embodiments, the patient's alkaline phosphatase level is about 1.5-3-fold greater than the upper limit of normal levels. Methods of determining the levels of these molecules are well known. Normal levels of ALT in the blood range from about 7-56 units/liter. Normal levels of AST in the blood range from about 10-40 units/liter. Normal levels of GGT in the blood range from about 9-48 units/liter. Normal levels of alkaline phosphatase in the blood range from about 53-128 units/liter for a 20- to 50-year-old man and about 42-98 units/liter for a 20- to 50-year-old woman.

Accordingly, in some embodiments, a solid dispersion disclosed herein, reduces level of AST, ALT, and/or GGT in an individual having elevated AST, ALT, and/or GGT levels. In some embodiments, the level of ALT is reduced at least 2-, at least 3-, at least 4-, or at least 5-fold. In some embodiments, the level of ALT is reduced about 2- to about 5-fold. In some embodiments, the level of AST is reduced at least 2-, at least 3-, at least 4-, or at least 5-fold. In some embodiments, the level of AST is reduced about 1.5 to about 3-fold. In some embodiments, the level of GGT is reduced at least 2, at least 3, at least 4, or at least 5-fold. In some embodiments, the level of GGT is reduced about 1.5 to about 3-fold.

In some embodiments, administration of a solid dispersion disclosed herein, to a subject results in a reduced NAFLD Activity Score (NAS). For example, in some embodiments, steatosis, inflammation, and/or ballooning is reduced upon treatment. In some embodiments, the compounds disclosed herein reduce liver fibrosis. In some embodiments, the compounds reduce serum triglycerides. In some embodiments, the compounds reduce liver triglycerides.

In some embodiments, the patient is at risk of developing an adverse effect prior to administering a solid dispersion disclosed herein. In some embodiments, the adverse effect is an adverse effect which affects the kidney, lung, heart, and/or skin. In some embodiments, the adverse effect is pruritus.

In some embodiments, the patient has had one or more prior therapies. In some embodiments, the liver disorder progressed during the therapy. In some embodiments, the patient has had one or more prior therapies with another FXR agonist other that Compound I. In some embodiments, the patient suffered from pruritus during at least one of the one or more prior therapies.

In some embodiments, the therapeutically effective amount is below the level that induces an adverse effect in the patient, such as below the level that induces pruritus, such as grade 2 or grade 3 pruritus.

### ENUMERATED EMBODIMENTS

1. A solid dispersion comprising a compound having the formula:

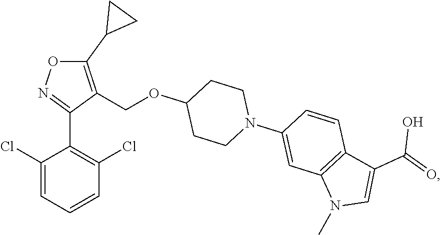 [SMILES: Cn1cc(C(=O)O)c2ccc(N3CCC(OCc4c(-c5c(Cl)cccc5Cl)noc4C4CC4)CC3)cc21]

wherein the compound is substantially amorphous and is dispersed in a polymer.
2. The solid dispersion of embodiment 1, wherein the polymer is a hydrophilic polymer.
3. The solid dispersion of embodiment 1 or 2, wherein the polymer is a homopolymer or a copolymer of a vinyl lactam.
4. The solid dispersion of any one of embodiments 1-3, wherein the polymer is a homopolymer or a copolymer of vinylpyrrolidone or vinylcaprolactam.
5. The solid dispersion of any one of embodiments 1-4, wherein the polymer is vinylpyrrolidone-vinyl acetate copolymer or vinylcaprolactam-vinyl acetate-ethylene glycol copolymer.
6. The solid dispersion of any one of embodiments 1-5, wherein the polymer is vinylpyrrolidone-vinyl acetate copolymer.
7. The solid dispersion of any one of embodiments 1-5, wherein the polymer is vinylcaprolactam-vinyl acetate-ethylene glycol copolymer.
8. The solid dispersion of any one of embodiments 1-7, wherein the weight ratio of the compound to the polymer is between about 1:1 and about 1:10.
9. The solid dispersion of embodiment 8, wherein the weight ratio of the compound to the polymer is about 1:3.
10. A pharmaceutical composition comprising the solid dispersion of any one of embodiments 1-9 and a pharmaceutically acceptable excipient.
11. The pharmaceutical composition of embodiment 10, wherein the pharmaceutically acceptable excipient comprises a diluent.
12. The pharmaceutical composition of embodiment 11, wherein the diluent comprises microcrystalline cellulose.
13. The pharmaceutical composition of any one of embodiments 10-12, wherein the pharmaceutically acceptable excipient comprises a disintegrant.
14. The pharmaceutical composition of embodiment 13, wherein the disintegrant comprises crospovidone.
15. The pharmaceutical composition of any one of embodiments 10-14, wherein the pharmaceutically acceptable excipient comprises a glidant.
16. The pharmaceutical composition of embodiment 15, wherein the glidant comprises colloidal silicon dioxide.
17. The pharmaceutical composition of any one of embodiments 10-16, wherein the pharmaceutically acceptable excipient comprises a lubricant.
18. The pharmaceutical composition of embodiment 18, wherein the lubricant comprises magnesium stearate.
19. The pharmaceutical composition of any one of embodiments 10-18, wherein the composition comprises between about 1 mg and about 30 mg of the compound.
20. The pharmaceutical composition of embodiment 19, wherein the composition comprises about 5 mg or about 25 mg of the compound.
21. The pharmaceutical composition of any one of embodiments 10-20, wherein the composition is in the form of a tablet.
22. A method of preparing the solid dispersion of any one of embodiments 1-9, comprising hot-melt extruding a mixture of the compound and the polymer.
23. The method of embodiment 22, wherein the hot-melt extrusion is performed at a temperature of between about 120° C. and about 180° C.
24. A method of treating a liver disorder in a subject in need thereof, comprising administering a therapeutically effective amount of the solid dispersion of any one of embodiments 1-9 or the pharmaceutical composition of any one of claims 10-21.
25. The method of embodiment 24, wherein the liver disorder is liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), or non-alcoholic steatohepatitis (NASH).

### Examples

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

### Example 1. Preparation of Solid Dispersions by Hot-Melt Extrusion

A mixture of 35 g Compound I and 105 g vinylpyrrolidone-vinyl acetate copolymer (sold under the trademark Kollidon® VA 64) (API:polymer, w/w=1:3), a mixture of 35 g Compound I and 105 g vinylcaprolactam-vinyl acetate-ethylene glycol copolymer (sold under the trademark Soluplus®) (API:polymer, w/w=1:3), and a mixture of 35 g Compound I and 105 g hydroxypropyl methylcellulose acetate succinate (HPMCAS-LG) (API:polymer, w/w=1:3) were hot-melt extruded on a Leistritz (ZSE 18HP) extruder. The extrusion conditions and properties of the resulting solid dispersions are provided in Table 1.

TABLE 1

| Mixture | Compound I/Kollidon ® VA 64 | | | Compound I/Soluplus ® | | | Compound I/HPMCAS-LG | |
|---|---|---|---|---|---|---|---|---|
| Temperature | 145 | 140 | 135 | 135 | 130 | 120 | 145 | 155 |
| (° C.) | | | | | | | | |
| Screw Speed | 130 | 130 | 131 | 111 | 111 | 100 | 100 | 100 |
| (RPM) | | | | | | | | |
| Feed Rate | 15 | 15 | 10 | 10 | 10 | 15 | 11 | 11 |
| (g/min) | | | | | | | | |
| Pressure | 3 | 3 | 3 | 3 | 3 | 3 | 3 | 4 |
| (Bar) | | | | | | | | |
| Appearance | a | a | a | a | a | b | c | c |
| of extrudate | | | | | | | | |

a: Transparent yellow solid
b: Opaque white solid
c: Opaque gray solid

The resulting extrudates were milled, sieved, and measured by differential scanning calorimetry (DSC). The DSC data suggests that Compound I was substantially amorphous in those solid dispersions.

Impurities in various samples (Compound I; a physical mixture of Compound I and Kollidon® VA 64; a physical mixture of Compound I and Soluplus®; Compound I/Kollidon® VA 64 solid dispersion; and Compound I/Soluplus® solid dispersion) were determined by High-performance Liquid Chromatography (HPLC). The results are provided in Table 2.

TABLE 2

| | | | Imp 1 | Imp 2 | Imp 3 |
|---|---|---|---|---|---|
| | | % | (%) | (%) | (%) |
| | Assay | Total | RRT = | RRT = | RRT = |
| Sample | % | IMP | 0.95 | 1.16 | 1.23 |
| Compound I | 99.4 | 0.26 | 0.06 | 0.15 | 0.04 |
| Compound I/VA 64 | 95.6 | 0.56 | 0.18 | 0.13 | 0.26 |
| solid dispersion | | | | | |
| (prepared at 145° C.) | | | | | |
| Compound I/VA 64 | 96.3 | 0.28 | 0.06 | 0.17 | 0.04 |
| physical mixture | | | | | |
| Compound I/Soluplus ® | 86.9 | 0.40 | 0.11 | 0.13 | 0.16 |
| solid dispersion | | | | | |
| (prepared at 135° C.) | | | | | |
| Compound I/Soluplus ® | 90.2 | 0.29 | 0.07 | 0.18 | 0.04 |
| physical mixture | | | | | |

### Example 2. Solubility Study

Compound I, Compound U/Kollidon® VA 64 solid dispersion prepared according to Example 1, and Compound U/Soluplus® solid dispersion prepared according to Example 1 were dissolved in phosphate buffer (pH=6.8) and the resulting mixtures were agitated at 37° C. Samples were taken at 15 minutes, 30 minutes, 60 minutes, 120 minutes, 4 hours, 8 hours, and 24 hours. Each sample was filtered and the resulting filtrate was measured by ultraviolet-visible spectroscopy to determine the concentration of Compound I. The results are provided in Table 3 As shown in Table 3, Compound I had low solubility in phosphate buffer. Compound I/Kollidon® VA 64 solid dispersion and Compound I Soluplus® solid dispersion both showed significantly improved solubility compared with Compound I. Compound I/VA 64 solid dispersion showed the best solubility.

TABLE 3

| | Concentration of Compound I (μg/mL) | | | | | | |
|---|---|---|---|---|---|---|---|
| Sample | 15 min | 30 min | 60 min | 120 min | 4 hrs | 8 hrs | 24 hrs |
| Compound I | 0.086 | 0.166 | 0.172 | 0.188 | 0.166 | 0.207 | 0.250 |
| Compound I/VA 64 | 36.66 | 46.62 | 53.01 | 62.72 | 69.06 | 75.81 | 69.64 |
| solid dispersion | | | | | | | |
| Compound I/VA 64 | 3.26 | 3.68 | 3.85 | 3.60 | 3.65 | 2.48 | 2.57 |
| physical mixture | | | | | | | |
| Compound I/Soluplus ® | 5.00 | 10.61 | 11.80 | 12.33 | 12.82 | 12.69 | 13.17 |
| solid dispersion | | | | | | | |
| Compound I/Soluplus ® | 1.97 | 2.95 | 3.67 | 3.51 | 4.11 | 4.06 | 4.91 |
| physical mixture | | | | | | | |

### Example 3. Bioavailability Study

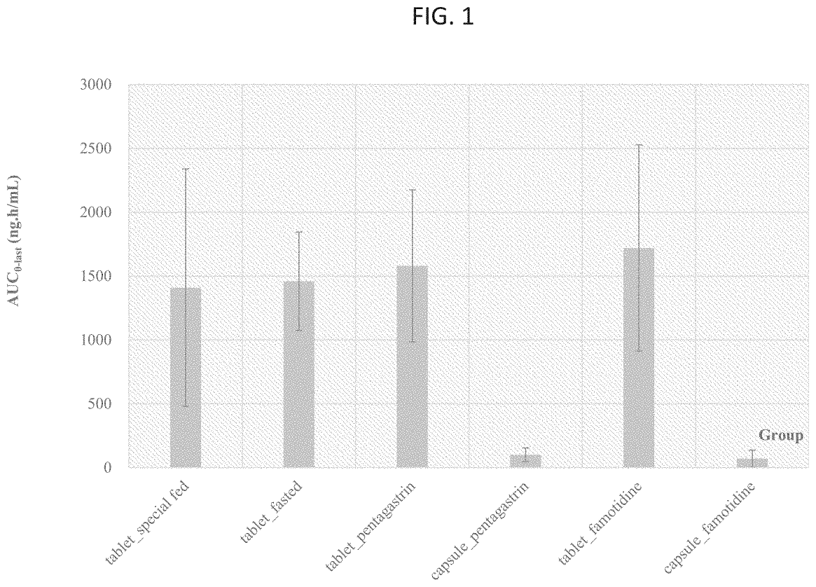

Compound I/Kollidon® VA 64 solid dispersion prepared according to Example 1 was blended with excipients. The mixture was compressed to manufacture tablets of 5-mg and 25-mg strengths. Compound I, which was not in a solid dispersion, was also blended with excipients to manufacture capsules of 25-mg strength. Both types of tablets showed good stability under accelerated and long-term conditions and dissolution results were well within the immediate release requirements of NLT 70% of the label claim of Compound I in 45 minutes. Tablets of 25-mg strength and capsules containing 25 mg of Compound I were tested in dog pharmacokinetic study. The results are shown in FIG. 1. Mean percent bioavailability (F %) of the tablet was between 30% and 40% under different pHs and feeding conditions; while the bioavailability (F %) of the capsule was only between 1.5% and 3%.

### Example 4. Bioavailability Study

A Phase 1 randomized, open-label study was conducted in 16 healthy subjects. PK samples of Compound I were obtained up to 72 hours after single oral administration of: 1) 5-mg strength and 25-mg strength tablets (prepared according to Example 3) in 16 fasted subjects; 2) 25-mg strength capsule (containing 25 mg Compound I not in a solid dispersion) in 8 fasted subjects; and 3) 5-mg strength tablet (prepared according to Example 3) in 8 subjects after a high-fat, high calorie meal. Safety and tolerability of the tablets and capsule were also assessed.

Compound I tablet was rapidly absorbed under the fasted condition; absorption was more gradual for Compound I capsule. Plasma Cₘₐₓ and AUC increased approximately dose proportionally in the range of 5 mg to 25 mg for the tablets. Systemic exposure of Compound I in fasted subjects was ˜4-fold higher for 25-mg strength tablet vs. 25-mg strength capsule. Food slowed Compound I tablet absorption, with increased Tₘₐₓ and decreased Cₘₐₓ relative to fasted, but did not significantly impact total systemic exposure. There were no notable trends in adverse events (AE) between treatment groups, across dose levels or formulation. Most AEs were mild in severity and considered not related or unlikely related to study drug.

Compound I solid dispersion tablets achieved faster absorption and higher systemic exposure compared to capsules containing Compound I. They can be administered without regard to food. Compound I was safe and well tolerated in all treatment groups.

All documents, including patents, patent application and publications cited herein, including all documents cited therein, tables, and drawings, are hereby expressly incorporated by reference in their entirety for all purposes.

While the foregoing written description of the solid dispersions, uses, and methods described herein enables one of ordinary skill in the art to make and use the solid dispersions, uses, and methods described herein, those of ordinary skill in the art will understand and appreciate the existence of variations, combinations, and equivalents of the specific embodiments, methods, and examples herein.

1. A pharmaceutical composition comprising a substantially amorphous form of the compound of formula (I)

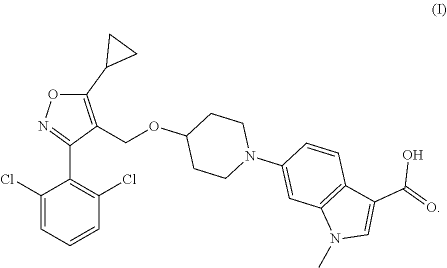 [SMILES: Cn1cc(C(=O)O)c2ccc(N3CCC(OCc4c(-c5c(Cl)cccc5Cl)noc4C4CC4)CC3)cc21]

2. The pharmaceutical composition of claim 1, wherein the composition comprises between about 1 mg and about 30 mg of the compound of formula (I).

3. The pharmaceutical composition of claim 1, wherein the composition comprises between about 5 mg and about 25 mg of the compound of formula (I).

4. The pharmaceutical composition of claim 1, wherein the composition comprises between about 5 mg and about 15 mg of the compound of formula (I).

5. The pharmaceutical composition of claim 1, wherein the composition comprises about 10 mg of the compound of formula (I).

6. The pharmaceutical composition of claim 1, wherein the composition comprises about 15 mg of the compound of formula (I).

7. The pharmaceutical composition of claim 1, wherein more than about 85% by weight of the compound of formula (I) in the composition is amorphous.

8. The pharmaceutical composition of claim 1, wherein more than about 95% by weight of the compound of formula (I) in the composition is amorphous.

9. The pharmaceutical composition of claim 1, wherein more than about 99% by weight of the compound of formula (I) in the composition is amorphous.

10. The pharmaceutical composition of claim 1, wherein the pharmaceutical composition is substantially free of a crystalline form of the compound of formula (I).

11. The pharmaceutical composition of claim 1, wherein the compound of formula (I) is dispersed in a polymer.

12. The pharmaceutical composition of claim 11, wherein the polymer is a hydrophilic polymer.

13. The pharmaceutical composition of claim 11, wherein the polymer is a homopolymer or a copolymer of a vinyl lactam.

14. The pharmaceutical composition of claim 11, wherein the polymer is a homopolymer or a copolymer of vinylpyrrolidone or vinylcaprolactam.

15. The pharmaceutical composition of claim 11, wherein the polymer is vinylpyrrolidone-vinyl acetate copolymer or vinylcaprolactam-vinyl acetate-ethylene glycol copolymer.

16. The pharmaceutical composition of claim 11, wherein the polymer is vinylpyrrolidone-vinyl acetate copolymer.

17. The pharmaceutical composition of claim 11, wherein the polymer is vinylcaprolactam-vinyl acetate-ethylene glycol copolymer.

18. The pharmaceutical composition of claim 11, wherein the weight ratio of the compound of formula (I) to the polymer is between about 1:1 and about 1:10.

19. The pharmaceutical composition of claim 18, wherein the weight ratio of the compound of formula (I) to the polymer is about 1:3.

20. The pharmaceutical composition of claim 1, wherein the pharmaceutical composition comprises the compound of formula (I) in a solid dispersion.

21. A method of treating non-alcoholic steatohepatitis (NASH) in a human patient in need thereof, comprising administering to the patient a pharmaceutical composition comprising a therapeutically effective amount of a substantially amorphous form of the compound of formula (I)

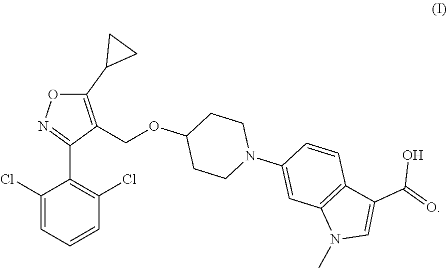 [SMILES: Cn1cc(C(=O)O)c2ccc(N3CCC(OCc4c(-c5c(Cl)cccc5Cl)noc4C4CC4)CC3)cc21]

22. The method of claim 21, wherein the composition comprises between about 1 mg and about 30 mg of the compound of formula (I).

23. The method of claim 21, wherein the composition comprises between about 5 mg and about 25 mg of the compound of formula (I).

24. The method of claim 21, wherein the composition comprises between about 5 mg and about 15 mg of the compound of formula (I).

25. The method of claim 21, wherein the composition comprises about 10 mg of the compound of formula (I).

26. The method of claim 21, wherein the composition comprises about 15 mg of the compound of formula (I).

27. The method of claim 21, wherein more than about 85% by weight of the compound of formula (I) in the composition is amorphous.

28. The method of claim 21, wherein more than about 95% by weight of the compound of formula (I) in the composition is amorphous.

29. The method of claim 21, wherein more than about 99% by weight of the compound of formula (I) in the composition is amorphous.

30. The method of claim 21, wherein the pharmaceutical composition is substantially free of a crystalline form of the compound of formula (I).

31. The method of claim 21, wherein the compound of formula (I) is dispersed in a polymer.

32. The method of claim 31, wherein the polymer is a hydrophilic polymer.

33. The method of claim 31, wherein the polymer is a homopolymer or a copolymer of a vinyl lactam.

34. The method of claim 31, wherein the polymer is a homopolymer or a copolymer of vinylpyrrolidone or vinylcaprolactam.

35. The method of claim 31, wherein the polymer is vinylpyrrolidone-vinyl acetate copolymer or vinylcaprolactam-vinyl acetate-ethylene glycol copolymer.

36. The method of claim 31, wherein the polymer is vinylpyrrolidone-vinyl acetate copolymer.

37. The method of claim 31, wherein the polymer is vinylcaprolactam-vinyl acetate-ethylene glycol copolymer.

38. The method of claim 31, wherein the weight ratio of the compound of formula (I) to the polymer is between about 1:1 and about 1:10.

39. The method of claim 38, wherein the weight ratio of the compound of formula (I) to the polymer is about 1:3.

40. The method of claim 31, wherein the pharmaceutical composition comprises the compound of formula (I) in a solid dispersion.
